(19) United States
(12) Patent Application Publication
(10) Pub. No.: US 2025/0299590 A1
(43) Pub. Date: Sep. 25, 2025

(54) INFORMATION OUTPUT METHOD, INFORMATION OUTPUT DEVICE, AND RECORDING MEDIUM

(71) Applicant: CASIO COMPUTER CO., LTD., Tokyo (JP)

(72) Inventors: Hiroshi SHIMIZU, Tokyo (JP); Nobuto YOSHIDA, Tokyo (JP); Ryunosuke ONO, Tokyo (JP); Masashi UEDA, Tokyo (JP); Katsunori ISHII, Tokyo (JP); Yunosuke AZUMA, Tokyo (JP); Hiroki TOMITA, Tokyo (JP)

(73) Assignee: CASIO COMPUTER CO., LTD., Tokyo (JP)

(21) Appl. No.: 19/088,317

(22) Filed: Mar. 24, 2025

(30) Foreign Application Priority Data
Mar. 25, 2024 (JP) 2024-047455

(51) Int. Cl. G09B 5/02; A63B 24/00; A63B 71/06

(52) U.S. Cl. CPC G09B 5/02; A63B 24/0075; A63B 71/0622

(57) ABSTRACT

An information output method to be executed by a computer includes: a setting operation to set a goal, a strategy, and a plan for an activity of a user, the strategy and the plan being for achieving the goal; and an outputting operation to output an instruction regarding the activity according to the set plan and a state of the user. The setting operation includes: a first setting operation to set a final goal in the activity and the strategy for achieving the final goal; and a second setting operation to set an interim goal and the plan for achieving the interim goal based on the strategy. The interim goal is set between the final goal and the user's initial state at a time of setting the final goal. The outputting operation outputs the instruction according to the plan for the interim goal and the state of the user.

### CROSS-REFERENCE TO RELATED APPLICATIONS

This application is based upon and claims the benefit of priority from the prior Japanese Patent Application No. 2024-047455, filed on Mar. 25, 2024, the entire contents of which are incorporated herein by reference.

### TECHNICAL FIELD

The present disclosure relates to an information output method, an information output device, and a recording medium.

### DESCRIPTION OF RELATED ART

There is a technology to evaluate actions or operations performed by a subject and show the next goal, improvement targets, or advice, based on the actions/operations. The contents of the evaluation may include quantitative results and evaluation values regarding performance in the process of achieving results. When setting goals, parameters such as the age, sex, height, and weight of the subject may be used as necessary, according to the type of actions. Japanese Unexamined Patent Application Publication No. 2005-108025 discloses a technology for vocational training. According to JP2005-108025A, parameters of high performers in the respective duties are set as targets; and advice and corrective action targets are output, based on the difference between the target parameters and the evaluation values of each subject, improvements of the subject in work, and so forth.

Examples of the actions and operations include hobbies and sports activities, as well as the above-mentioned duties. There are also mobile device applications and smart watches that output appropriate goals and advice for improving an outcome, based on a user's running posture, movements, and the resulting speed and time required.

### SUMMARY OF THE INVENTION

The present disclosure relates to an information output method to be executed by a computer, including: a setting operation to set a goal, a strategy, and a plan for an activity of a user, the strategy and the plan being for achieving the goal; and an outputting operation to output an instruction regarding the activity according to the set plan and a state of the user, wherein the setting operation includes: a first setting operation to set a final goal in the activity and the strategy for achieving the final goal; and a second setting operation to set an interim goal and the plan for achieving the interim goal based on the strategy, the interim goal being set between the final goal and an initial state of the user at a time of setting the final goal, wherein the outputting operation outputs the instruction according to the plan for the interim goal and the state of the user.

### BRIEF DESCRIPTION OF DRAWINGS

FIG. 1 illustrates a configuration of an information output system according to an embodiment.

FIG. 2A is a block diagram illustrating a functional configuration of a measurement device.

FIG. 2B is a block diagram illustrating a functional configuration of an electronic device.

FIG. 3 is a block diagram illustrating a functional configuration of a server device.

FIG. 4 is a table showing a list of processes for supporting practice.

FIG. 5 is a flowchart of a control procedure of a method setting process of this embodiment.

FIG. 6 is a flowchart of a control procedure of a plan setting process.

FIG. 7 is a flowchart of a control procedure of an advice control process.

FIG. 8 is a flowchart of a control procedure of an advice output control process to be executed by the measurement device.

FIG. 9 is a diagram illustrating an example of intervention of an assist process.

### DETAILED DESCRIPTION

Hereinafter, an embodiment of the present disclosure is described with reference to the figures.

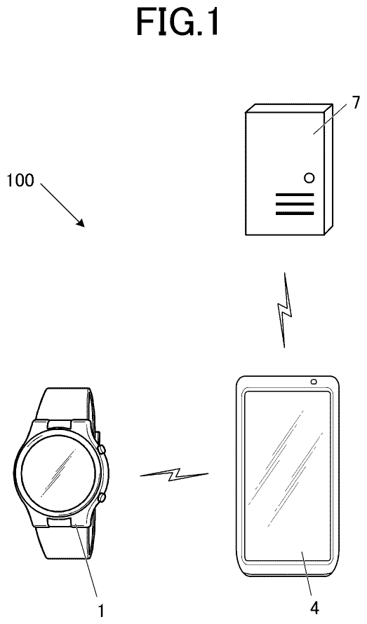

FIG. 1 is a diagram illustrating a configuration of an information output system 100 according to this embodiment. The information output system 100 of this embodiment creates plans for a user to achieve a goal in a certain activity (e.g., running). The information output system 100 can support the user regarding contents of the plans, execution of the plans, the state in achieving the goal, and tasks by providing the user with explanation, notification of the state, and advice.

The information output system 100 includes a measurement device 1, an electronic device 4, and a server device 7.

The measurement device 1 is a body-worn terminal device that is worn on the user's body to perform measurement on the activity. Herein, the measurement device 1 is a wristwatch-type device, such as a smart watch or an activity meter. There may be two or more measurement devices 1. For example, a waist-worn measurement device 1 may be included in addition to the wristwatch-type measurement device 1. The measurement device 1 receives manipulations related to selection, start, and stop of the activity and performs measurement. The measurement device 1 may be configured to perform simple parameter detection processing in real time during measurement and after measurement and output simple measurement information or advice, for example.

The electronic device 4 obtains measurement results by the measurement device 1 and analyzes the measurement results. The electronic device 4 can display advice, comments, and so forth based on the analysis results, together with the analysis results. The electronic device 4 is, for example, a smartphone. The measurement device 1 and the electronic device 4 may be constantly connected for a long time for communications. For example, the measurement device 1 and the electronic device 4 may be communicatively connected by the low energy standard of Bluetooth (registered trademark).

The server device 7 obtains analysis results from the electronic device 4 of each user and stores the analysis results as a database 732. The server device 7 performs statistical processing of the analysis results stored in the database 732 and obtains information on a large number of users, such as the distribution of abilities and the tendency of ability improvements by training. The electronic device 4 of each user can refer to these pieces of information, based on the level of the user, for example. The electronic device 4 is communicatively connected to the server device 7 via the Internet. For the communication connections, a wireless LAN and/or a mobile phone line may be used, for example.

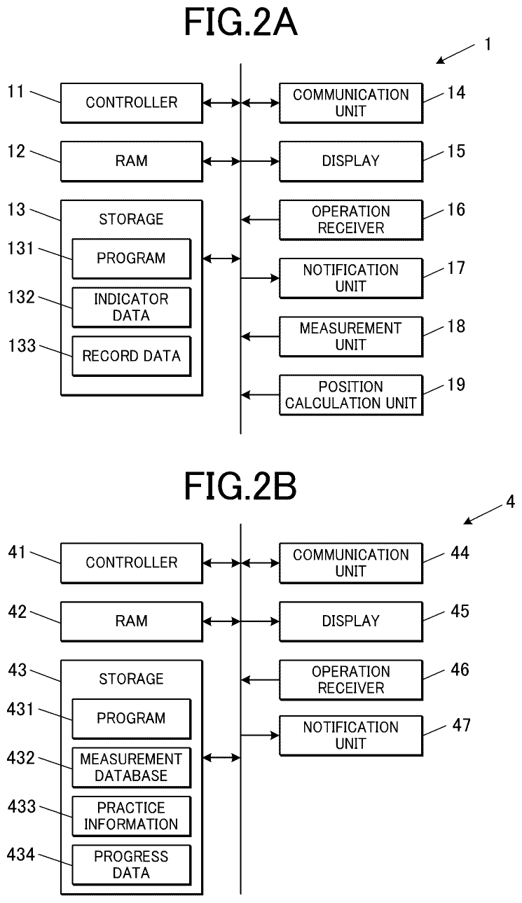

As illustrated in the block diagram of FIG. 2A, the measurement device 1 includes a controller 11, a random-access memory (RAM) 12, a storage 13, a communication unit 14, a display 15, an operation receiver 16, a notification unit 17, a measurement unit 18, and a position calculation unit 19.

The controller 11 includes a processor that controls overall operations of the measurement device 1. The processor may be a general-purpose central processing unit (CPU) or a dedicated microcomputer. There may be one or more processors. A single processor may centrally perform entire processing, or multiple processors may perform processing in parallel or independently for their respective purposes.

The RAM 12 provides a working memory space for the processor of the controller 11 and stores temporary data. The RAM 12 is, for example, a DRAM.

The storage 13 is a nonvolatile memory. The storage 13 stores a program 131, setting data, record data 133, and so forth. The record data 133 includes records of results of analyzing measurement results of past activities. The measurement data on activities may be transferred from the RAM 12 to the storage 13 and stored therein for an appropriate period until the data is sent to the electronic device 4. The nonvolatile memory may be a flash memory, for example. The setting data may include indicator data 132 that indicates a target goal and references of parameters (indicator values) corresponding to the target goal. The parameters may include part or all of values widely known as running parameters, such as a stride, a pitch, a tilt or sway of the upper body, the magnitude of arm swing, the rotation of the hip position, and the magnitude of left/right sway and up/down movements, for example. The record data 133 is obtained from the electronic device 4 after the electronic device 4 ends analysis.

The communication unit 14 includes a module that controls communication operations. As described above, the communication unit 14 can be communicatively connected to the electronic device 4 over Bluetooth, for example.

The display 15 includes a digital display screen. Under the control of the controller 11, the display 15 controls display on the digital display screen. The display 15 may be configured to display various parameters related to the activity and temporal changes of the parameters in real time or as records after the activity ends. The display 15 may be configured to display the current date and time together with the various parameters or switch the display between the current date and time and the parameters.

The operation receiver 16 includes one or more push button switches. The operation receiver 16 detects a press of the push button switch and outputs a detection signal to the controller 11. The operation receiver 16 may include a crown or a touchscreen. The operation receiver 16 detects the pulling out, pushing back, and rotating of the crown and outputs operation signals corresponding to the detection to the controller 11. The touchscreen is overlaid on the digital display screen. The operation receiver 16 detects a touch on the touchscreen and outputs a signal indicating the touched position to the controller 11.

The notification unit 17 performs notification to the outside. The notification may be generation of beeps, generation of vibration, or output of sounds, for example. The notification unit 17 has a configuration corresponding to the type of notification. The sounds may be output by a speaker. In addition to or instead of a speaker, earphones may output the sounds in response to receiving audio signals. The earphones may be wireless earphones that receive audio signals via the communication unit 14 over Bluetooth, convert the signals into sounds, and output the sounds, for example.

The measurement unit 18 performs various kinds of measurement of the activity. The measurement unit 18 includes, for example, an acceleration sensor, an orientation sensor, and a pulse sensor. In addition to these sensors, the measurement unit 18 may include part or all of a gyroscope sensor, a temperature sensor, an atmospheric pressure sensor, an oxygen saturation sensor, and so forth. The measurement results by the measurement unit 18 are converted into digital discrete values at predetermined sampling frequencies corresponding to the respective pieces of measurement data and are output to the controller 11.

The position calculation unit 19 receives radio waves from a positioning satellite of the Global Navigation Satellite System (GNSS) and calculates the position, based on the reception results. The position calculation unit 19 may include a module dedicated to receiving radio waves and calculating the position.

As illustrated by the block diagram of FIG. 2B, the electronic device 4 includes a controller 41, a RAM 42, a storage 43, a communication unit 44, a display 45, an operation receiver 46, and a notification unit 47.

The controller 41 includes a processor that controls overall operations of the electronic device 4. The processor may be a general-purpose central processing unit (CPU) or a dedicated microcomputer. There may be one or more processors. A single processor may centrally perform entire processing, or multiple processors may perform processing in parallel or independently for their respective purposes.

The RAM 42 provides a working memory space for the processor of the controller 41 and stores temporary data. The RAM 42 may be, for example, a DRAM. The temporary data includes measurement results during the activity obtained from the measurement device 1. The measurement results may be deleted after the analysis process ends.

The storage 43 is a nonvolatile memory. The storage 43 stores a program 431, setting data, measurement database 432, and so forth. The measurement database 432 includes analysis results of measurement results of the activity in the past. The nonvolatile memory may be a flash memory, for example. The setting data includes practice information 433 and plan progress data 434 regarding the activity. The program 431 may include individual programs of a method setting process, a plan setting process, an advice control process, and an assist process, which are described later.

The communication unit 44 includes a module for controlling communication operations. As described above, the communication unit 44 can be communicatively connected to the measurement device 1 over Bluetooth and can communicate with the server device 7 over the Internet, for example. The communication unit 44 may obtain weather information around the current location from outside, such as information on rainfall, snowfall, wind direction, and wind strength, for example.

The display 45 includes a digital display screen. Under the control of the controller 41, the display 45 controls display on the digital display screen. The display 45 may also be configured to display information on functions other than information on the activity.

The operation receiver 46 includes, for example, a touchscreen and a push button switch. The touchscreen is overlaid on the digital display screen. The operation receiver 46 detects a touch on the touchscreen and outputs a signal indicating the touched position to the controller 41. The operation receiver 46 detects a press of the push button switch and outputs a detection signal to the controller 41.

The notification unit 47 performs notification to the outside. The notification may be generation of a beep, generation of vibration, or output of sounds, for example. The notification unit 47 has a configuration corresponding to the type of notification.

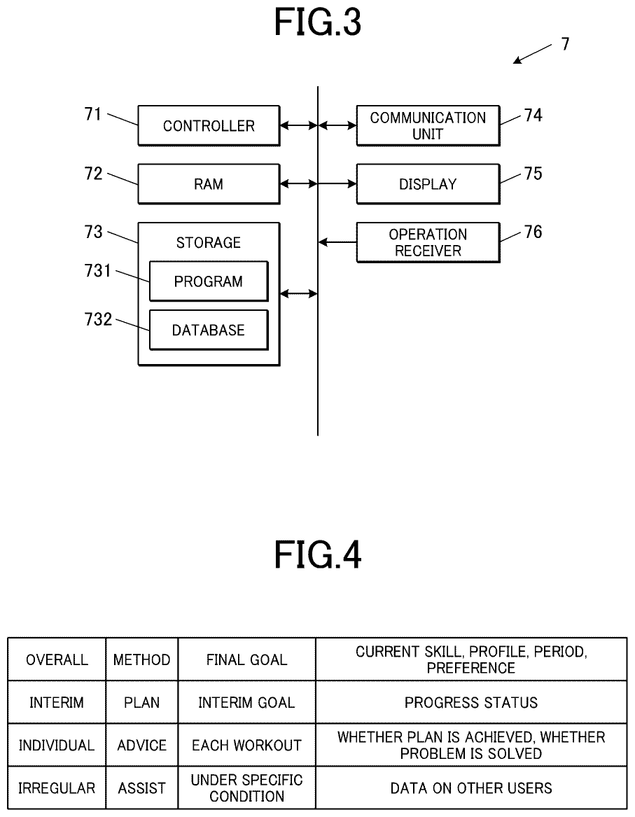

FIG. 3 is a block diagram illustrating a functional configuration of the server device 7. The server device 7 may be a normal personal computer (PC), for example. The server device 7 includes a controller 71, a RAM 72, a storage 73, a communication unit 74, a display 75, and an operation receiver 76.

The controller 71 includes a processor that controls overall operations of the server device 7. The processor may be a general-purpose CPU or a dedicated microcomputer. A single processor may centrally perform entire processing, or multiple processors may perform processing in parallel or independently for their respective purposes.

The RAM 72 provides a working memory space for the processor of the controller 71 and stores temporary data. The RAM 72 may be, for example, a DRAM.

The storage 73 is a nonvolatile memory and stores a program 731, a database 732, and so forth. The nonvolatile memory may be, for example, a hard disk drive (HDD) or a flash memory. The storage 73 may be an auxiliary storage device externally attached to the main body of the PC. For another example, the storage 73 may include a network drive or a cloud server provided on a network.

The communication unit 74 includes a module that controls communication operations. As described above, the communication unit 74 can communicate with the electronic device 4 over the Internet.

The display 75 includes a digital display screen. Under the control of the controller 71, the display 75 controls display on the digital display screen.

The operation receiver 76 may include a push button switch, a pointing device such as a mouse, and a keyboard, for example.

The display 75 and the operation receiver 76 may be peripheral devices attached to the PC main body that includes the controller 71, the RAM 72, and the storage 73 (and the communication unit 74). For another example, the server device 7 may be basically accessed via an external terminal device, and the display 75 and so forth except the power button and the reset switch may not be normally connected to the server device 7.

The database 732 stores profiles of a large number of users, methods used by the users in the past, implementation periods of the methods, performance records by the implementation of the methods, changes in parameters, and so forth. The database 732 may also store information on troubles that can occur during implementation of methods, points where users may face difficulties in improving their abilities, states of suspending implementation of methods halfway, and goal achievement rates, for example. Herein, a method refers to a determined way, strategy, or scheme for achieving a goal and/or object. Examples of a method include: Method A (technique, physical, mental), Method B (technique, physical, tactics), and Method C (technique, physical, mental, tactics). A method may include items other than the above items (technique, etc.) defined for each method.

Next, the operation for supporting a user in achieving a goal is described.

According to the information output method of this embodiment, a practice plan is determined, based on the user's target goal and target date. A practice plan is a plan of active workouts for the user to develop his/her abilities and gain skills. Herein, the server device 7 as an information output device of this embodiment creates a practice plan for the user as an example. The GUI operation for creating a practice plan may be performed on the electronic device 4. In this case, the electronic device 4 sends information necessary for creating a practice plan to the server device 7, and the server device 7 creates the practice plan. The created practice plan is stored on the server device 7 and sent to the electronic device 4. For another example, the electronic device 4 may obtain information necessary for creating a practice plan from the server device 7 and creates the practice plan. The measurement device may receive user's manipulations for creating a practice plan.

By following such a practice plan, the user can improve his/her abilities and skills more efficiently toward a goal, as compared with doing daily haphazard practice. However, if there is a large gap between the user's ability and the target goal, the user may not see the path to the target goal and may not maintain motivation. The information output method of this embodiment includes a first setting operation to set a target goal as a final goal and an overall strategy (method) for achieving the final goal. Thereafter, the information output method of this embodiment includes a second setting operation to set an appropriate interim goal(s) between the user's current ability and the final goal and set a more detailed practice plan for each interim goal. By following instructions that are output based on the practice plan, the user sequentially achieves the interim goals and finally achieves the final goal. The next goal and plan may be determined each time an interim goal at each stage is achieved.

FIG. 4 is a table showing a list of processes for supporting the user in achieving goals.

The final goal in the overall strategy (method) is determined, based on the initial state of abilities and skills at the time the final goal is determined, the user's profile, the target time until achieving the final goal, and so forth. The target time may include a specific period of time, such as 300 hours or 90 days, and/or a specific date, such as Dec. 1, 2024, for example. The final goal of a user may be determined based on data collected from a large number of users in the past. Specifically, the final goal may be determined within a range of final goals that have been achieved by a certain percentage of other similar users or more, for example. The similar users are users whose initial states (abilities, skills, etc.) are within a predetermined reference range from the initial state of the user. The profile may include the user's physical characteristics (e.g., height and weight), age, and sex. Based on the overall final goal, the method is determined as the overall strategy of practice. A method may be selectable when the method is socially known to a certain extent and when the number of users (samples) who have been successful by implementing the method is equal to or greater than a reference. A newly proposed method (including an improvement of an existing method) may be tested by test users and, based on the information from the test users, the applicability of the method to ordinary users may be determined. In this embodiment, multiple candidate methods may be stored beforehand in association with the respective ranges of final goals. When there are multiple candidate methods, the user may select one method according to his/her preference.

The final goal may be expressed by the performance desired by the user. In running, the performance may be a combination of the running distance and the running time, for example. The running distance may include the total distance that the user run in a certain term. The running time may include the total time of running in a certain term.

The path from the initial state to the final goal is divided into sections; and interim goals are determined for the respective sections according to expected improvements in the method. A plan is determined stepwise and in detail for achieving each interim goal in accordance with the method. There may be one or more interim goals. The interim goal may be determined based on abilities or skills. The interim goal may be determined based on abilities or skills expected in each period (e.g., one to two weeks or one month). A plan may include details about strengthening specific body parts and improvement points, based on skills and abilities expected at each stage. The details of a plan corresponding to the set method may be determined by output of a machine learning model, for example. For example, training data may be generated by an expert, based on output plans corresponding to an input method or input parameters at the start of the method; and a machine learning model may be trained with the training data. Such a machine learning model may have a decision tree structure, for example.

The interim goals and plans for achieving the interim goals may not directly lead to the final goal. In such a case, the user may not see the meaning of doing workouts in accordance with the plan. Therefore, in updating interim goals and setting new plans for the interim goals, the meaning and effectiveness of the interim goals and plans may be explained.

In setting a plan, a plan that has been set can be adjusted if there is a discrepancy between abilities/skills expected by workouts and actual improvements by the workouts. A plan for achieving an interim goal may include a detailed menu for achieving the interim goal (e.g., practice contents, training contents). For example, a plan may include contents for training the physical aspect included in Method A.

In each workout to be done according to the set plan, it is determined whether a set goal, such as an interim goal, has been achieved. Further, the performance of the user is compared with the performance corresponding to the target level of the plan, and specific advice is output, based on the extracted problems. That is, the problems may be extracted, based on an expected target level.

Thus, in this embodiment, the final goal, the interim goals, and the individual workouts are hierarchically separated and processed, whereas the processes in the adjacent stages of the hierarchy have an organic connection. Therefore, the information output method of this embodiment maintains the user's motivation by encouraging the user to challenge a close goal and immediate improvement points and efficiently guides the user to the final goal.

Even with the above, individual users may stagnate at certain levels or have difficult problems to overcome. The information output method of this embodiment further includes an assist process that irregularly intervenes when such a specific condition is met. The assist process quantitatively evaluates what the other users have achieved according to the effective method. That is, the assist process is called as a single-purpose interrupt process when a specific condition is met irregularly. When the user stagnates in a specific condition, the assist process allows the user to understand that the goal has been achieved statistically in many cases. The assist process thus contributes to maintaining the user's motivation. The assist process may not be called on a nonscheduled basis. The assist process may be performed after every workout so that the user can regularly receive assists reflecting the workout result.

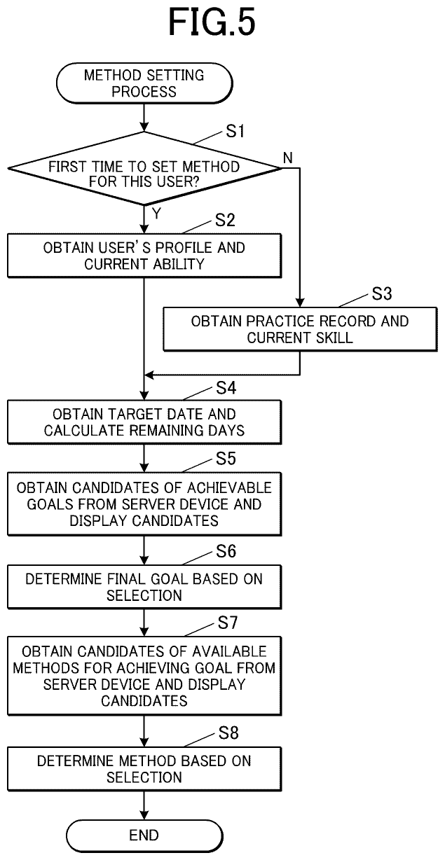

FIG. 5 is a flowchart of a control procedure of a method setting process of this embodiment.

The method setting process, which serves as a first setting operation in the program of this embodiment, may be executed once when the user starts an activity toward a goal.

The controller 71 determines whether this is the first time to create a method of the activity for the user (S1). When determining that this is the first time to create a method of this activity for the user (S1: Y), the controller 71 obtains the user's profile and current abilities (S2). If, for example, the user has been registered at the time of setting the application in the electronic device 4, the registration information of the user may be obtained as the profile. The current abilities may be obtained by obtaining and analyzing the actual running results of the user. The controller 71 then proceeds to step S4.

When this is not the first time to create a method of this activity for this user (S1: N), the controller 71 obtains information on practice records until now and skills of the user (S3). The controller 71 then proceeds to step S4.

The controller 71 obtains information on the target date (deadline) for achieving the goal and calculates the target time (remaining days) from the current date to the target date (S4). Typically, the target date for achieving the goal is set to the date of an event, such as a tournament or competition. The target date may be earlier than such an event date or may be simply determined as desired by the user. The target date may be received by the operation receiver 16 or 46, or the like.

The controller 71 extracts selectable candidates for the final goal. The candidates for the final goal are in a range assumed to be achievable by the user with a certain level of achievability or higher, based on the current abilities, skills, and the target time of the user. The display 75 displays a list of the extracted candidates of the final goal (S5). For another example, display control information of the extracted candidates may be sent to the electronic device 4 via the communication unit 74, and the display 45 of the electronic device 4 may display the list of the extracted candidates. Thus, the user is not allowed to select a realistically unachievable goal. The higher the goal is, the harder the workouts will become to achieve the goal. The controller 71 detects the user's selection operation on the operation receiver 76 or receives contents of the selection operation on the operation receiver 46 via the communication unit 74. Based on the selection, the controller 71 determines the final goal (S6).

The controller 71 obtains candidate methods that can be used to achieve the selected final goal and displays a list of the candidate methods on the display 75 or on the display 45 (S7). As described above, there may be multiple candidate methods. Multiple candidate methods may partly or entirely differ from each other in the following: the number of possible workouts per week, the duration of each workout, and so forth. Since users are in various situations regarding work, study, family, and so forth, each user may select a feasible method, based on his/her own living conditions and motivation. The number of possible workouts per week may be included in the initially input conditions along with the target date. The controller 71 determines the method, based on the selection operation on the operation receiver (S8). The determined method is sent to the electronic device 4, and the electronic device 4 stores the determined method as practice information 433 together with the information on the final goal and the target date. If the user finds none of the methods on the list feasible, the user may perform an operation to cause the controller 71 to return to step S5 and select a lower final goal. The controller 71 ends the method setting process.

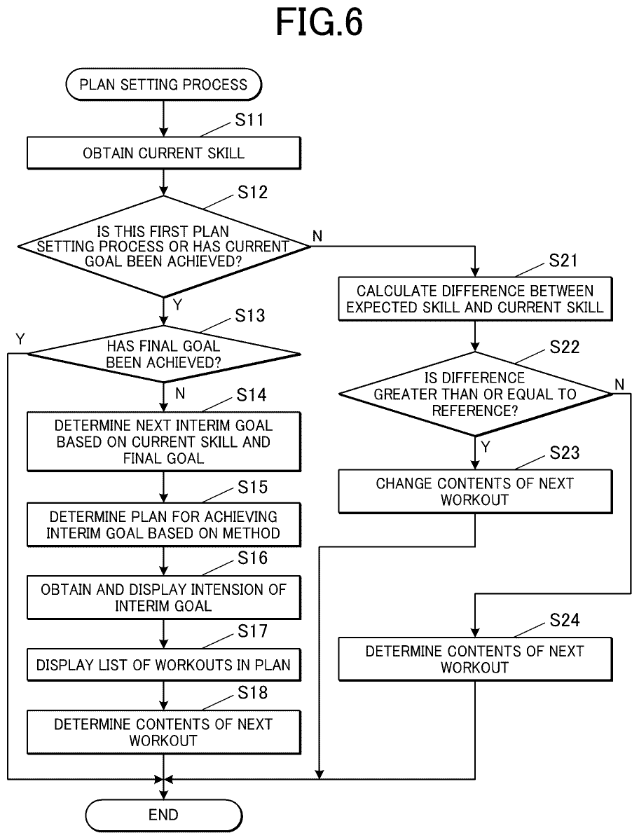

FIG. 6 is a flowchart of a control procedure of a plan setting process.

The plan setting process, which serves as a second setting operation in the program of this embodiment, is executed after the method setting process or after each workout, for example.

The controller 71 obtains the current skills and abilities (S11). The controller 71 determines whether or not (i) this is the first plan setting process for the set method and the final goal or (ii) the currently set goal (interim goal or final goal) has been achieved (S12).

When determining that (i) this is the first plan setting process for the set method and the final goal or (ii) the currently set goal has been achieved (S12: Y), the controller 71 determines whether the achieved current goal is the final goal (S13). When determining that the achieved goal is the final goal (S13: Y), the controller 71 ends the plan setting process. That is, the controller 71 does not set a new goal or plan. However, if the final goal is achieved earlier than the target goal, such a plan may be set that allows the user to keep the state of achieving the final goal until the target date.

When determining that the achieved goal is not the final goal (S13: N), the controller 71 determines the next interim goal, based on the current skills, abilities and the final goal (S14). When the interim goal is determined based on a period (e.g., the number of days), the interim goal can be flexibly determined based on expected improvements in abilities and skills during the period. As described above, each interim goal may not correspond to an equal division of the ability or skill growth range toward the final goal. For example, an interim goal may be aimed at improving a specific skill among multiple skills first. The subsequent interim goals may be aimed at improving the other skills or abilities. The controller 71 determines a plan corresponding to the interim goal according to the set method (S15). The interim goal and the plan are sent to the electronic device 4 via the communication unit 74. The electronic device 4 stores the received interim goal and plan in the practice information 433.

The controller 71 sends the intention and effectiveness of the set interim goal to the electronic device 4 and causes the electronic device 4 to display the intention and effectiveness on the display 45 (S16). Thus, the user can clearly understand the purpose of workouts to achieve the interim goal. Explanations of intentions and effectiveness may be stored beforehand in the server device 7 in association with parameters to be improved toward interim goals. The controller 71 may send the contents of the plan of workouts having been set so far to the electronic device 4 and cause the display 45 to display a list of the contents (S17). The controller 71 sets the practice contents of the next workout in the plan and sends the practice contents to the electronic device 4 (S18). The electronic device 4 stores the received contents in the practice information 433 (S18). The controller 71 then ends the plan setting process.

In the determination process of step S12, when determining that (i) this is not the first plan setting process for the set method and the final goal and (ii) the current goal has not been achieved (S12: N), the controller 71 proceeds to step S21. In step S21, the controller 71 calculates the difference between an expected change in skill/ability by workouts until now, particularly the most recent workout, and the actual change in skill/ability (S21). The controller 71 determines whether or not the calculated difference is greater than or equal to a reference (S22). When skill and ability include multiple items, the controller 71 determines whether or not the difference regarding any one of the items is greater than or equal to a reference. For another example, the controller 71 may determine whether or not a combination of differences regarding multiple items is greater than or equal to a reference by using a machine learning model. In this case, the differences regarding multiple items may be flexibly weighted, based on the importance of the items.

When determining that the calculated difference is not greater than or equal to a reference (S22: N), the controller 71 determines practice contents of the next workout, based on the plan of workouts that was set when the previous goal was achieved. The controller 71 sends the set practice contents to the electronic device 4 via the communication unit 74, and the controller 41 of the electronic device 4 stores the received practice contents in the practice information 433 (S24). The controller 71 then ends the plan setting process. When determining that the calculated difference is greater than or equal to a reference (S22: Y), the controller 71 adjusts the practice contents of the normally set workout by adding changes to the practice contents, based on the difference (S23). That is, the controller 71 changes the contents of the plan so as to cover delays in improvement of ability and obtainment of skill. The details of the change may be determined by the intervening assist process, which is described later. The changed practice contents are stored in the practice information 433. The controller 71 then ends the plan setting process.

The steps of the method setting process and the plan setting process correspond to the setting operation of this embodiment.

In each workout, the user is guided according to the plan created as described above, and the user learns the movements that he/she should make.

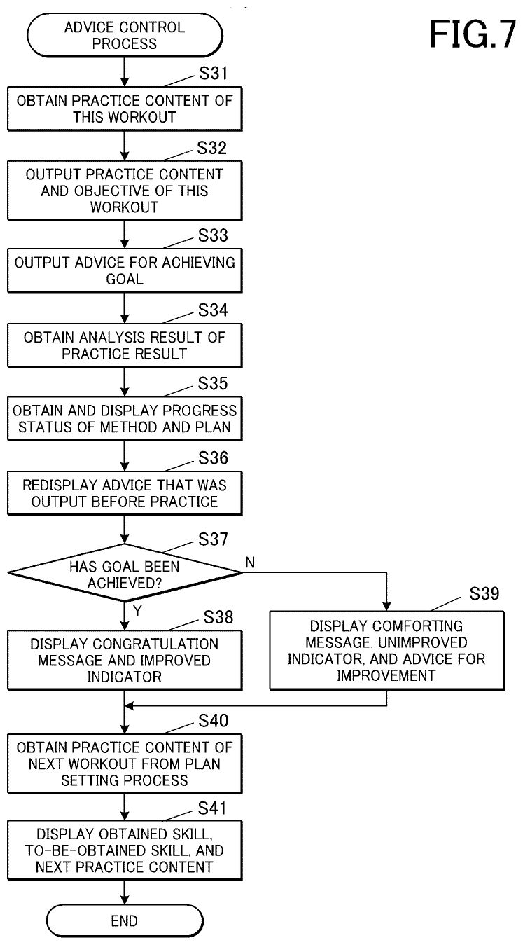

FIG. 7 is a flowchart of a control procedure of an advice control process.

The advice control process starts when the user is about to perform a workout.

The controller 71 obtains the practice contents of this workout (S31). The controller 71 outputs the obtained practice content and its objective (S32). The obtained practice content and its objective may be output to the display 45 of the electronic device 4 or to the measurement device 1 via the communication units 74 and 44 so as to be displayed on the measurement device 1. Output sentences may be partially a combination of fixed phrases and the contents. For another example, sentences to be output may be stored beforehand in the storage 73 in association with practice contents. When the practice contents are set, the electronic device 4 may obtain sentences corresponding to the practice contents from the server device 7 and display the sentences. When the practice contents have been set at the end of the previous workout, the set practice contents may be changed depending on the weather and the user's physical condition at the time of this time workout. For example, the set practice contents may be changed by including practice contents that are to be set in the next or later workout. For another example, if the user is not in good physical condition, the practice contents may be reduced.

The controller 71 outputs advice for achieving the goal (S33). The output advice may include generalities or may focus on obtained problems or weaknesses of the user. When the advice is relatively long, machine learning model may be used to generate natural sentences. The machine learning model may be Transformer using deep learning, for example. Such machine learning can create sentences in a natural style using slightly different expressions for an identical content every time advice is output. As a result, the user is less likely to treat the comment lightly, and any of the expressions may help the user intuitively grasp a solution to his/her problem. Thereafter, when the user starts the workout, the measurement device 1 sequentially obtains measurement results. The controller 11 of the measurement device 1 or the controller 41 of the electronic device 4 may detect low parameters during the workout, based on the measurement results and indicator data 132, and may output advice corresponding to the detection. The measurement device 1 or the electronic device 4 may sequentially obtain wording of the advice from the server device 7 or may obtain possible wording beforehand from the server device 7. When the measurement device 1 is constantly connected to the electronic device 4 for communication, the measurement device 1 may appropriately send the measurement data to the electronic device 4 in real time or at certain time intervals. If the communication connection is cut off during a workout, the measurement device 1 sends all the measurement results to the electronic device 4 after the workout ends. The measurement results may not be the measurement data itself obtained by the measurement device 1 but may be the above-mentioned parameter values converted from the measurement data. After the workout ends, the workout is analyzed, based on the obtained parameter values and so forth. The controller 71 obtains the analysis result from the electronic device 4 (S34). Instead, the controller 71 of the server device 7 may obtain parameter values and so forth from the electronic device 4 and analyze the workout. The obtained analysis result is stored in the database 732. The analysis result is also stored in the measurement database 432 of the electronic device 4.

Based on the analysis result, the controller 71 obtains the progress status of the method and plan and sends the obtained content to the electronic device 4; and the electronic device 4 displays the progress status on the display 45 (S35). The electronic device 4 stores the obtained progress status in the progress data 434. The progress data 434 may also be sent to the measurement device 1 and stored in the record data 133. The controller 71 redisplays the contents of the advice that were displayed before the start of the workout (S36). The controller 71 determines whether the currently set goal has been achieved (S37).

When determining that the goal has been achieved (S37: Y), the controller 71 sends, to the electronic device 4, the indicators that have been improved by the achievement along with a message congratulating the achievement; and the electronic device 4 displays the message and the indicators on the display 45 (S38). The controller 71 then proceeds to step S40. When determining that the goal has not been achieved (S37: N), the controller 71 sends, to the electronic device 4, the indicators that have not been improved or low indicators along with a comforting message for performing the workout; and the electronic device 4 displays the indicators and the message on the display 45 (S39). The controller 71 also causes the display 45 of the electronic device 4 to display advice for improving these indicators. The controller 71 then proceeds to step S40.

In step S40, the controller 71 obtains the practice contents of the next workout that are determined by the plan setting process executed at the end of the workout (S40). The controller 71 outputs the obtained skills, the skills to be obtained in the future, and the next practice contents to the electronic device 4 and causes the display 45 to display them (S41). The controller 71 ends the advice control process.

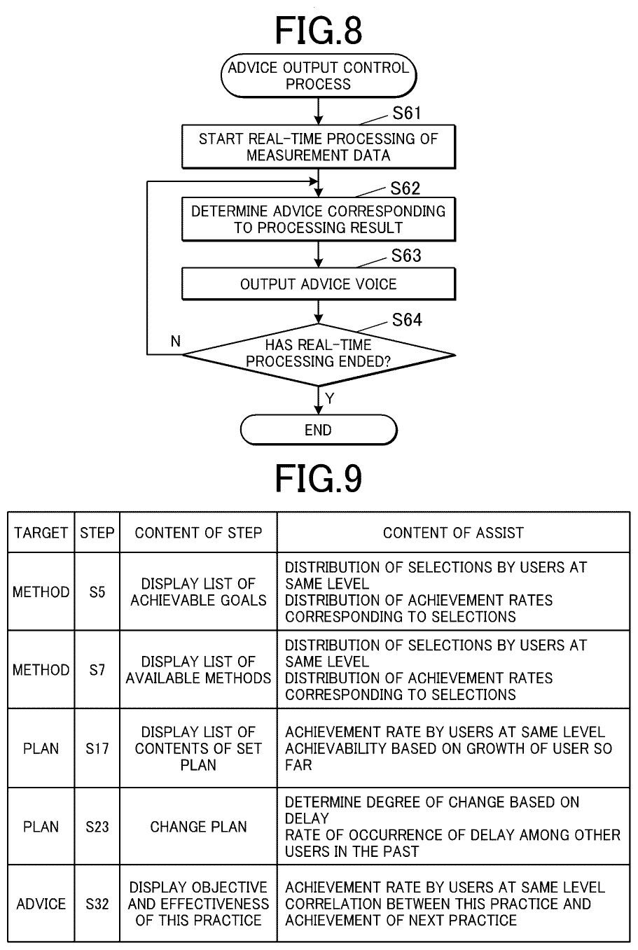

FIG. 8 is a flowchart of a control procedure of the advice output control process that is executed by the measurement device 1. The advice output control process is executed in real time by the measurement device 1 during measurement of a workout. That is, the advice output control process is executed between steps S33 and S34 of the advice control process.

The controller 11 starts parameter detection processing in real time along with measurement by the measurement unit 18 and the position calculation unit 19 (S61). The controller 11 determines advice, based on parameters obtained by the processing (S62). The controller 11 causes the notification unit 17 to output sound corresponding to the contents of the advice (S63). As described above, wireless earphones may output the sound by receiving signals via the communication unit 14. The advice output herein may include information on movements, posture, speed, pitch, and/or stride, for example.

If there is no particular advice to be given, the controller 11 may set “No advice” in step S62 and skip step S63. The controller 11 determines whether real-time inputting and processing of measurement t data has been completed (S64). When determining that inputting and processing of measurement data has not been completed (S64: N), the controller 11 returns to step S61. When determining that inputting and processing of measurement data has been completed (S64: Y), the controller 11 ends the advice output control process.

Next, conditions for the assist process to intervene are described.

FIG. 9 is a diagram illustrating an example of intervention of the assist process.

There may be a case where the user (target user) does not understand an optimal final goal or method in setting a method and so forth. In such a case, the assist process of this embodiment can provide assist information to the target user. The assist information relates to selections and results of other similar users who have close profiles and levels to the target user. Whether the profiles/levels of other users are close to this user may be determined, based on an appropriate reference. For example, differences among combinations of multiple parameters may be expressed by Euclidean distances in a multidimensional space. When the Euclidean distance regarding the profile/level of a user is less than a reference value, the user may be regarded as an other similar user who is within a reference range from the target user. The assist process may also provide distribution of selection tendencies indicating selections of other similar users or a distribution of achievement statuses indicating how many users achieved the selected goal as a vicariously experience, for example. The assist process may also output a message indicating a specific achievable goal, such as “80% or more of runners at around your level can achieve the following final goal . . . ”.

The assist process of this embodiment can also provide, to the user, assist information indicating how long it took for other users at the same level to achieve the interim goal and the achievement rate of the interim goal. Although some interim goals may seem to include highly difficult contents at first glance, achievement rates of such interim goals are not necessarily low if a user follows plans of a proven method. Therefore, providing information regarding the achievement rate and the period required to achieve a goal can prevent a decrease in motivation. Further, based on the improvement rate of the user's abilities so far and the user's state, it is possible to estimate the achievability of a goal and the time required to achieve the goal to some extent. Providing such information allows the user to believe that the goal is achievable, to increase self-efficacy, and to mentally and specifically prepare for the time required to achieve the interim goal.

On the other hand, as described above, there may be a case where the user does not improve abilities or acquire skills as expected even though the user has carried out workouts according to the set plan. In such a case, the plan may be changed based on the degree of discrepancy between the actual improvement and an expected improvement obtained from the practice information 433 and the progress data 434. The plan may be changed to cover delays and push the user toward the goal. Alternatively, the plan may be changed to accept delays and relax the interim goal to avoid decreasing the user's motivation by presenting practice beyond the user's desired level. In the latter case, changing the plan can affect achievement of the final goal. Therefore, to avoid ultimately diminishing the user's motivation to improve his/her abilities, the timing and pattern of providing information may be appropriately controlled in consideration of the possibility that the final goal is changed. In this case, the user may be provided with the percentage of other users who fell behind in the past and the percentage of those other users who caught up afterwards. For example, a message such as “the probability of achieving the final goal is about 80% by runners at around your current level” may be displayed to maintain and improve motivation of the user.

Further, in the advice control at each workout, the achievement rate of users at the same level may be displayed along with the purpose and effectiveness of the workout. In addition, the correlation between the goal achievement rate of the current practice and the goal achievement of the next practice may be displayed. Detailed aspects, such as individual practice contents, may not directly lead to improvement of performance. By presenting the correlation between such practice contents and the next practice contents regarding the goal achievement rate, it is possible to increase the user's motivation to achieve the goal.

The output control operation in the above steps S32, S33, S38, S39, S41, and so forth and the output contents in the assist process correspond to the outputting operation and output means of this embodiment.

### Other Embodiments

In the above, an activity in which the user is supported for achieving a goal is exercise, particularly running, as an example. However, the activity is not limited to running. For example, the activity may be other kinds of exercise, such as cycling, swimming, trail running, or walking. The activity may not be exercise. For example, the present disclosure may be applied to making a plan for achieving a goal in learning. As for the type of learning, it is preferable that the learning state be easily monitorable. Examples of learning that can be easily monitored include: learning of basic kanji, words, phrases and proverbs; learning of calculations involving basic transformations of equations; and rote learning. In learning calculations, fill-in-the-blank questions in calculation processes may be included to promote understanding of the user and to detect points that are difficult for the user to understand.

In such a case, the information output system 100 may not include the measurement device 1. Instead, the electronic device 4 may store an online learning application that provides explanations and study questions online and that identifies the user's answers on the operation receiver 46, for example. The operation receiver 46 may include an external touchpad and a touch pen.

For example, in arithmetic or mathematics calculations, a target unit to be focused in calculation practices and the final goal in application-level formula transformations may be determined, based on information on the level of understanding and proficiency of each field (unit) and information on a test date. Various patterns and levels of calculation questions in the determined target unit are sequentially set in a plan.

The assist process may insert advice corresponding to patterns of errors in calculation practices regarding each interim goal. The assist process may also show partial hints for answer patterns that the user does not understand well.

In this case, the assist process may provide the user with the correct answer rate by users at the same level, the goal achievement rate as a result of learning by the method, and so forth.

Other examples of a non-exercise activity may include board games, such as shogi and go. The information output method of the present disclosure is suitably applicable to an activity that is performed by manipulating an electronic device and that can be quantitatively evaluated. The information output method of the present disclosure may also be applicable to training for personal development in workplaces.

As described above, the information output method of this embodiment is executed by a computer and includes the following: (1) the setting operation to set a goal for a certain activity of the user and a plan for achieving the goal; and (2) the outputting operation to output instructions related to the activity according to the set plan and the state of the user. The setting operation includes the following: (1a) the first setting operation to set a final goal in the activity and a strategy for achieving the final goal; (1b) the second setting operation to set an interim goal and a plan for achieving the interim goal, based on the strategy. The interim goal is set between the final goal and the initial state of the user at the time of setting the final goal. In the outputting operation, the instructions are output for each set interim goal according to the plan and the state of the user. According to the information output method, the computer can output strategic advice according to the user's changing state and progress in a medium-term to long-term plan toward the goal, as well as sequentially outputting advice corresponding to each state as in the known art. Thus, the information output method allows the user to deepen his/her understanding of the plans for achieving the goal and efficiently supports the user to improve his/her abilities. Further, based on the overall strategy for achieving the final goal, specific interim goals and plans for achieving the respective interim goals are determined. Such interim goals and plans allow the user to feel a sense of accomplishment of goals, and the user is further motivated toward the goals. Further, the user can get a more detailed understanding of the progress toward the goals.

In the first setting operation, a range of selectable final goals may be determined, based on the target time for achieving the final goal; and the final goal may be determined, based on the selection by the user. Achieving a goal often involves a target date (deadline). Based on such a deadline, the final goal may be determined within the range of goals achievable by the user, so that the user can keep motivation toward the goal. Thus, the information output method of this embodiment can support the user in improving his/her abilities and acquiring skills toward the final goal.

The range of goals may be determined, based on information of users who achieved their final goal and who have an initial state within a reference range from the initial state of the user. It is assumed that, if a goal was achieved by other users at the same level in the past, the goal is realistically achievable by the user. Since the user can recognize that the goal is realistically achievable, the user can actively engage in workouts. Thus, the information output method can effectively support the user in achieving the final goal.

In the first setting operation, a strategy may be selected from strategies that are stored beforehand in association with the final goal. There can be various methods (strategies) for workouts, depending on the type of activity and the ability to be improved for achieving the final goal. Strategies (methods) that have been proven effective to a certain extent may be selected and stored beforehand, so that the user can engage in workouts with confidence in the proposed strategy and plan. Accordingly, the user is more likely to achieve the final goal.

In the second setting operation, multiple interim goals may be set. Since the process to the final goal is divided into multiple sections and interim goals are set for the respective sections, the intervals between interim goals set by the user can be shortened. Thus, the information output method can further motivate and guide the user to achieve the goal.

Further, when the user achieves an interim goal, in the second setting operation, the next goal and the plan for achieving the next goal may be determined. According to the above, the interim goals and the plans for achieving the interim goals are not fixed at the initial stage; the next interim goal and plan are determined after an interim goal is achieved. Thus, the information output method can support the user more flexibly. By creating a plan according to the individual progress of the user, the information output method can further decrease the possibility that the user drops out before achieving the final goal.

In the second setting operation, when the next goal is set, an explanation of the intention and effectiveness of the plan for the next goal may be output. When multiple interim goals are set toward the final goal, these interim goals may have different contributions to improving performance toward the final goal. Specifically, some interim goals may not directly contribute to improving performance toward the final goal. In such cases, by explaining the purpose and effectiveness of each interim goal in advance, the information output method can reduce anxiety of the user and encourage the user to engage in the workouts more certainly.

In the information output method of this embodiment, changes in the user's state in the activity may be estimated; and the plan for the goal may be adjusted, based on the difference between the amount of the estimated change and the amount of the actual change in the activity. Practice plans based on methods are expected to yield results. However, there may be cases where plans do not yield sufficient results as generally expected, depending on the profile of the user. In such cases, in the information output method, the amount or the load of workouts may be increased to cover the difference between the actual result and the expected result; or the amount or the load of workouts may be reduced to avoid overdoing workouts. Thus, the information output method can effectively support users in achieving their goals by tailoring plans for the individual users.

Further, reference indicators regarding the activity may be determined according to the state of the user; and, when the indicators on the state of the user are lower than the reference indicators, advice for improving target points may be output.

Thus, the information output method may also output advice sequentially as in the known art. The advice may include contents based on the progress of the user toward his/her goal. Therefore, the information output method can appropriately provide necessary information to the user and support the user in achieving his/her goal.

Further, when the user selects a target goal or a method to be set, the information processing method of this embodiment may output (i) information on selection tendencies of similar users who performed the activity in the past and whose states were within a reference range from the state of the user and (ii) information on the similar users regarding the status of achievement of the final goal. There are cases where a user does not understand how to determine the final goal based on his/her abilities and what strategy he/she should follow in performing workouts. In such cases, the information output method presents, to the user, information on past settings of similar users at the same level and information on whether the similar users have achieved their final goals based on the settings. Thus, the information output method can effectively support the user in making selections.

According to this embodiment, the server device 7 as the information output device includes the controller 71. The controller 71 performs the setting operation of setting a goal for a certain activity of the user, a strategy, and a plan for achieving the goal. The controller 71 performs the outputting operation to output instructions related to the activity according to the set plan and the state of the user. As the setting operation, the controller 71 performs the following two setting operations: (1a) the first setting operation to set a final goal in the activity and a strategy for achieving the final goal; (1b) the second setting operation to set an interim goal and a plan for achieving the interim goal, based on the strategy. The interim goal is set between the final goal and the initial state of the user at the time of setting the final goal. In the outputting operation, the controller 71 outputs instructions for each set interim goal according to the plan and the state of the user. Such a server device 7 can output advice according to the user's changing state and progress in the plans toward the goal, as well as sequentially outputting advice corresponding to each state. Thus, the server device 7 allows the user to deepen his/her understanding of the plans for achieving the goal and efficiently supports the user to improve his/her abilities.

Further, with the program 731 of this embodiment, the processing relating to the information output method can be easily executed through software by a computer, such as the server device 7.

The above embodiment is not intended to limit the present disclosure and can be variously modified.

For example, although interim goals and corresponding plans are set in addition to the final goal in the above description, the plan setting process for setting one plan for the final goal may be executed in parallel with the advice control process. Conversely, an interim goal may be further divided into sub-goals, and more detailed sub-plans may be determined.

Further, the assist process may not be executed by interrupting the method setting process, the plan setting process, and the advice control process. The contents of the assist process may be included in the method setting process, the plan setting process, and the advice control process.

Further, although the assist process in the above embodiment is for presenting statistical contents mainly based on measurement results of other users in the past, the assist process is not limited to this.

Changing a set plan and practice schedules may not be based on the difference between expected improvements and actual improvements of skills/abilities in each workout.

A plan or practice schedule may be changed, based on tendencies of improvement rates in multiple workouts.

Although a plan is set every time an interim goal is achieved, the present disclosure is not limited to this. Plans for all stages may be set at the beginning. Even in such a case, plans and practice schedules may be changed or adjusted according to the actual improvement rate of the user's ability.

In the above description, the target time is fixed and the final goal is selected and determined. However, the present disclosure is not limited to this. The set target time may have a range. Further, methods that require time slightly exceeding the set target time may be included in selectable options.

In the above embodiment, the controller 71 of the server device 7 executes the advice control process before and after a workout, and the controller 11 of the measurement device 1 executes the advice output control process during the workout. However, the present disclosure is not limited to this. The controller 41 of the electronic device 4 may provide advice to the user during workouts (exercise, study).

Further, the operation of the server device 7 as the information output device described above may be distributed to and executed by multiple electronic devices, such as multiple server devices 7 or the server device 7 and the electronic device 4. Specifically, in a case where a device owned by the user (e.g., the electronic device 4) is not constantly connected to the server device 7 for communications, part of the processing of the server 7 may be appropriately distributed to the electronic device 4. Thus, necessary information can be timely provided to the user.

In the above description, the storage 73 includes a nonvolatile memory, such as a HDD or a flash memory, as an example of a computer-readable medium for storing the program 731 related to the goal achievement support of the present disclosure. However, the computer-readable medium is not limited to these. The computer-readable medium can be a different type of nonvolatile memory, such as a magneto-resistive random access memory (MRAM), or a portable recording medium, such as a CD-ROM or a DVD. Further, a carrier wave may be used as a medium to provide data of the program of the present disclosure via a communication line.

The detailed configurations, contents and orders of the steps in the processes, and so forth shown in the above embodiment can be appropriately modified without departing from the scope of the present disclosure. The scope of the present disclosure includes the scope of the invention described in the claims and their equivalents.

1. An information output method to be executed by a computer, comprising:
a setting operation to set a goal, a strategy, and a plan for an activity of a user, the strategy and the plan being for achieving the goal; and
an outputting operation to output an instruction regarding the activity according to the set plan and a state of the user,
wherein the setting operation includes:
a first setting operation to set a final goal in the activity and the strategy for achieving the final goal; and
a second setting operation to set an interim goal and the plan for achieving the interim goal based on the strategy, the interim goal being set between the final goal and an initial state of the user at a time of setting the final goal,
wherein the outputting operation outputs the instruction according to the plan for the interim goal and the state of the user.

2. The information output method according to claim 1, wherein in the first setting operation,
a range of selectable final goals is set, based on a target time for achieving the final goal, and
the final goal is set, based on a selection by the user.

3. The information output method according to claim 2, wherein
the range of selectable final goals is set, based on information of persons who previously achieved final goals and whose initial states were within a reference range from the initial state of the user.

4. The information output method according to claim 1, wherein in the first setting operation, the strategy is selected from strategies that selectable are stored beforehand in association with the final goal.

5. The information output method according to claim 1, wherein in the second setting operation, multiple interim goals are set.

6. The information output method according to claim 1, wherein in the second setting operation, in a case where the user achieves the interim goal, a next goal and the plan for achieving the next goal are set.

7. The information output method according to claim 6, wherein in the second setting operation, in a case where the next goal is set, an explanation of an intention and effectiveness of the plan for achieving the next goal is output.

8. The information output method according to claim 1, further comprising:
estimating a change in the state of the user in the activity; and
adjusting the plan for the achieving the goal, based on a difference between an amount of the estimated change and an amount of an actual change in the activity.

9. The information output method according to claim 1, further comprising:
determining references of indicators regarding the activity according to the state of the user; and
outputting advice for improving an indicator regarding the state of the user among the indicators in a case where the indicator is lower than a corresponding reference among the references.

10. The information output method according to claim 2, further comprising:
in receiving the selection by the user, outputting (i) information on selection tendencies of persons who previously performed the activity and whose states were within a reference range from the state of the user and (ii) information on whether the persons achieved the final goal.

11. The information output method according to claim 4, further comprising:
in receiving a selection by the user, outputting (i) information on selection tendencies of persons who previously performed the activity and whose states were within a reference range from the state of the user and (ii) information on whether the persons achieved the final goal.

12. An information output device comprising a processor that performs:
a setting operation to set a goal, a strategy, and a plan for an activity of a user, the strategy and the plan being for achieving the goal; and
an outputting operation to output an instruction regarding the activity according to the set plan and a state of the user,
wherein the setting operation includes:
a first setting operation to set a final goal in the activity and the strategy for achieving the final goal; and
a second setting operation to set an interim goal and the plan for achieving the interim goal based on the strategy, the interim goal being set between the final goal and an initial state of the user at a time of setting the final goal,
wherein in the outputting operation, the processor outputs the instruction according to the plan for the interim goal and the state of the user.

13. A non-transitory computer-readable storage medium storing a program that causes a computer to perform:
a setting operation to set a goal, a strategy, and a plan for an activity of a user, the strategy and the plan being for achieving the goal; and
an outputting operation to output an instruction regarding the activity according to the set plan and a state of the user,
wherein the setting operation includes:
a first setting operation to set a final goal in the activity and the strategy for achieving the final goal; and
a second setting operation to set an interim goal and the plan for achieving the interim goal based on the strategy, the interim goal being set between the final goal and an initial state of the user at a time of setting the final goal,
wherein in the outputting operation, the computer outputs the instruction according to the plan for the interim goal and the state of the user.
